(19) United States
(12) Patent Application Publication
(10) Pub. No.: US 2026/0256480 A1
(43) Pub. Date: Sep. 3, 2026

(54) THROMBECTOMY DEVICES AND METHODS

(71) Applicant: BARD PERIPHERAL VASCULAR, INC., Franklin Lakes, NJ (US)

(72) Inventors: Kyle J. HULL, Phoenix, AZ (US); Kristen HUNN, Gilbert, AZ (US); Tyler LENT, Mesa, AZ (US); Zachary WILLIS, Mesa, AZ (US); Danielle M. WORGER, Mesa, AZ (US)

(73) Assignee: BARD PERIPHERAL VASCULAR, INC., Franklin Lakes, NJ (US)

(21) Appl. No.: 19/164,135

(22) PCT Filed: Apr. 3, 2023
(86) PCT No.: PCT/US2023/017260

§ 371 Date: Sep. 10, 2025

(51) Int. Cl. A61B 17/22; A61B 17/00

(52) U.S. Cl. CPC A61B 17/22; A61B 17/00234; A61B 2017/00305; A61B 2017/00526; A61B 2017/00539; A61B 2017/00778; A61B 2017/22038; A61B 2017/22079; A61B 2217/005; A61B 2217/007

(57) ABSTRACT

A thrombectomy device includes a first catheter shaft, a second catheter shaft, a third catheter shaft, a first helical impeller, and a second helical impeller. The second catheter shaft is positioned within a first lumen of the first catheter shaft, wherein an outflow path is between the first catheter shaft and the second catheter shaft. The second catheter shaft includes an inflow opening. The first catheter shaft distal end defines an outflow opening positioned proximal to the inflow opening. The third catheter shaft is positioned within the second lumen, wherein an inflow path configured to direct material in a proximal direction is defined between the second catheter shaft and the third catheter shaft. The first helical impeller is positioned in the outflow path. The second helical impeller is positioned within the inflow path and configured to draw the fluid into the inflow opening.

### TECHNICAL FIELD

The present specification generally thrombectomy devices and methods and more particularly to thrombectomy devices and methods which include helical impellers.

### BACKGROUND

Thrombosis is a condition wherein a blood clot, known as a thrombus, is formed within a blood vessel, thus obstructing the normal blood flow through the blood vessel. Interventional medical procedures may generally include using thrombolytics to aid in extraction of the clot. However, use of thrombolytics on their own may lead to higher risks of bleeding and/or longer hospitals stays. Mechanical aspiration devices for removing clots are also known as opposed to using thrombolytics. However, such devices may become clogged or may be inefficient at removing clots.

Accordingly, a need exists for devices and methods for removing clots that, if using thrombolytics, reduces unwanted bleeding and/or is configured to reduce device blockage to improve aspiration and procedure efficiency.

### SUMMARY

Embodiments of the present disclosure provide improved thrombectomy devices and methods by providing quicker clot removal with improved recovery.

In one embodiment, a thrombectomy device includes a first catheter shaft, a second catheter shaft, a third catheter shaft, a first helical impeller, and a second helical impeller. The first catheter shaft defines a first lumen and has a first catheter shaft distal end. The second catheter shaft is positioned within the first lumen, wherein an outflow path configured to direct fluid in a distal direction is defined between the first catheter shaft and the second catheter shaft. The second catheter shaft defines a second lumen and includes a second catheter shaft distal end defining an inflow opening. The first catheter shaft distal end defines an outflow opening positioned proximal to the inflow opening. The third catheter shaft is positioned within the second lumen, wherein an inflow path configured to direct material in a proximal direction is defined between the second catheter shaft and the third catheter shaft. The first helical impeller is positioned in the outflow path configured to direct the fluid through the outflow path to the outflow opening. The second helical impeller is positioned within the inflow path, the second helical impeller configured to draw the fluid released from the outflow opening distally into the inflow opening, the second helical impeller having a diameter greater than an inner diameter of the outflow path.

In another embodiment, a thrombectomy device includes a first catheter shaft, a second catheter shaft, a third catheter shaft, a first helical impeller, and a second helical impeller, and a guidewire. The first catheter shaft defines a first lumen and has a first catheter shaft distal end. The second catheter shaft is positioned within the first lumen, wherein an outflow path configured to direct fluid in a distal direction is defined between the first catheter shaft and the second catheter shaft. The second catheter shaft defines a second lumen and includes a second catheter shaft distal end defining an inflow opening. The first catheter shaft distal end defines an outflow opening positioned proximal to the inflow opening. The third catheter shaft is positioned within the second lumen and defines a guidewire lumen, wherein an inflow path configured to direct material in a proximal direction is defined between the second catheter shaft and the third catheter shaft. The first helical impeller is positioned in the outflow path configured to direct the fluid through the outflow path to the outflow opening. The second helical impeller is positioned within the inflow path, the second helical impeller configured to draw the fluid released from the outflow opening distally into the inflow opening, the second helical impeller having a diameter greater than an inner diameter of the outflow path. The guidewire is configured to be positioned within the guidewire lumen.

In yet another embodiment, a method for assembling a thrombectomy device includes positioning a first catheter shaft defining a first lumen and comprising a first catheter shaft distal end about a second catheter shaft such that the second catheter shaft is positioned within the first lumen. An outflow path configured to direct fluid in a distal direction is defined between the first catheter shaft and the second catheter shaft, the second catheter shaft defining a second lumen and including a second catheter shaft distal end defining an inflow opening. The first catheter shaft distal end defines an outflow opening positioned proximal to the inflow opening. The method further includes positioning a third catheter shaft within the second lumen, wherein an inflow path configured to direct material in a proximal direction is defined between the second catheter shaft and the third catheter shaft, positioning a first helical impeller in the outflow path, the first helical impeller configured to direct the fluid through the outflow path to the outflow opening, and positioning a second helical impeller within the inflow path, the second helical impeller configured to draw the fluid released from the outflow opening distally into the inflow opening, the second helical impeller having a diameter greater than an inner diameter of the outflow path.

In yet another embodiment, a method of using a thrombectomy device includes positioning the thrombectomy device at a target site. The thrombectomy device includes a first catheter shaft defining a first lumen and comprising a first catheter shaft distal end, a second catheter shaft positioned within the first lumen, wherein an outflow path configured to direct fluid in a distal direction is defined between the first catheter shaft and the second catheter shaft, the second catheter shaft defining a second lumen and comprising a second catheter shaft distal end defining an inflow opening, wherein the first catheter shaft distal end defines an outflow opening positioned proximal to the inflow opening, a third catheter shaft positioned within the second lumen, wherein an inflow path configured to direct material in a proximal direction is defined between the second catheter shaft and the third catheter shaft, a first helical impeller positioned in the outflow path configured to direct the fluid through the outflow path to the outflow opening, and a second helical impeller positioned within the inflow path, the second helical impeller configured to draw the fluid released from the outflow opening distally into the inflow opening, the second helical impeller having a diameter greater than an inner diameter of the outflow path. The method further includes rotating the first helical impeller to move the fluid in the distal direction through the outflow path, and rotating the second helical impeller to initiate suction through the inflow path.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1A schematically depicts a thrombectomy system including a thrombectomy device, according to one or more embodiments shown and described herein;

FIG. 1B schematically depicts a longitudinal cross-section of the thrombectomy device of FIG. 1A, according to one or more embodiments shown and described herein;

FIG. 1C schematically depicts a head portion of the thrombectomy device of FIG. 1B, according to one or more embodiments shown and described herein;

FIG. 1D schematically depicts a distal end of the thrombectomy device of FIG. 1B, according to one or more embodiments shown and described herein;

FIG. 2A schematically depicts flow through the thrombectomy device of FIG. 1A-1D, according to one or more embodiments shown and described herein;

FIG. 2B schematically depicts flow through the thrombectomy device proximal to a distal end of the thrombectomy device of FIG. 1A-1D, according to one or more embodiments shown and described herein;

FIG. 3A schematically depicts a longitudinal cross-section of a thrombectomy device, according to one or more embodiments shown and described herein;

FIG. 3B schematically depicts a side view of the thrombectomy device of FIG. 3A, according to one or more embodiments shown and described herein;

FIG. 4 illustrates a flowchart depicting a method for assembling a thrombectomy device, according to one or more embodiments shown and described herein;

FIG. 5 illustrates a flowchart depicting a method of using a thrombectomy device, according to one or more embodiments shown and described herein; and

FIG. 6 schematically depicts a thrombectomy procedure with a thrombectomy device, according to one or more embodiments shown and described herein.

### DETAILED DESCRIPTION

Embodiments of the present disclosure are directed to thrombectomy devices, systems, and methods for removal of clots or other target lesions, such as within the venous and/or arterial system. In one embodiment, a thrombectomy device includes a plurality of coaxial shafts defining an outflow path configured to direct fluid in a distal direction and an inflow path configured to direct fluid and/or clot material in a proximal direction. A first helical impeller is positioned within the outflow path and a second helical impeller is positioned within the inflow path. In embodiments, as fluid is pushed through the outflow path in contacts the first helical impeller thereby causing the impeller to rotate, which may in turn cause the second helical impeller to rotate and aspirate or pull fluid and/or clot material into the inflow path. The pushing and pulling of fluid into and out of the thrombectomy device may create a local vortex operable to macerate clot material and remove the clot via the inflow path. These and additional benefits and embodiments will be described in greater detail below.

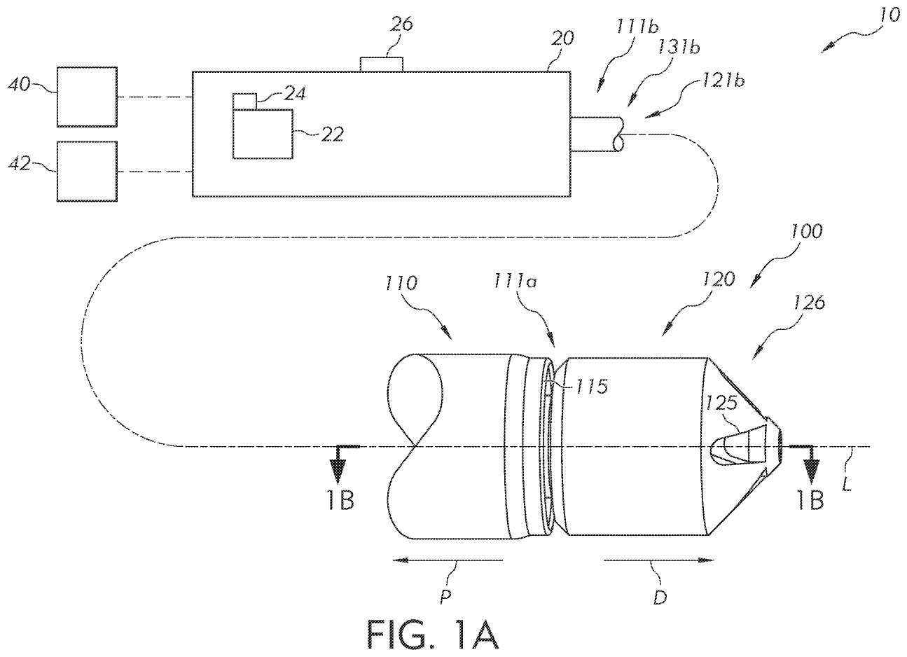

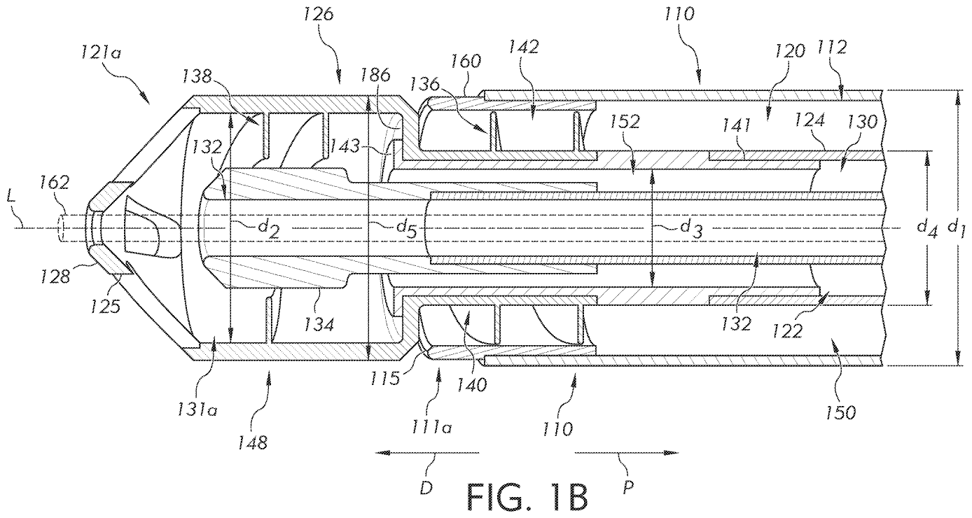

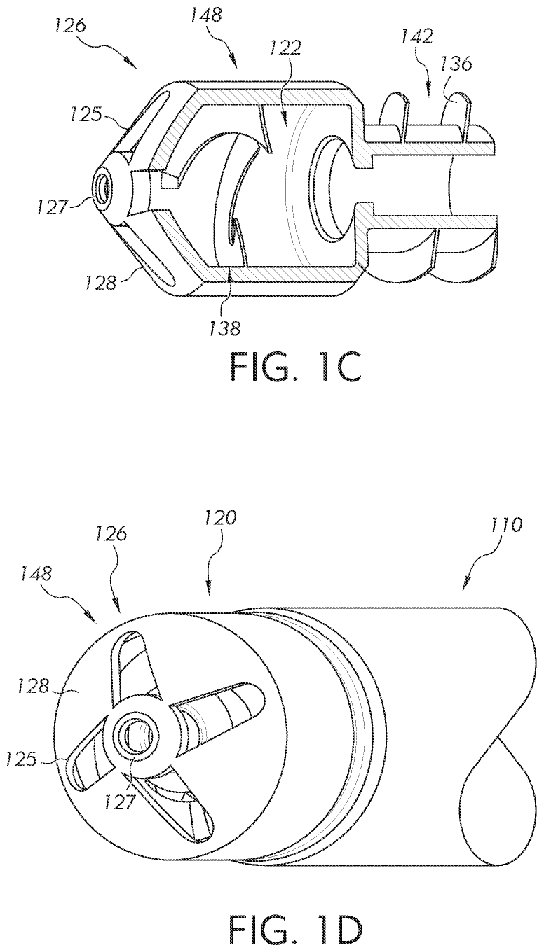

Referring to FIG. 1A, a thrombectomy system 10 is schematically depicted. The thrombectomy system 10 may generally include a thrombectomy device 100, which may be coupled to a handle 20 for handling by an operator.

Incorporated into the handle 20 may be a controller 22. In other embodiments, the controller 22 may be separate from a handle 20. The controller 22 may include any number of processors, chips, memories (storing non-transitory computer-readable instructions) or the like and may control operation of the thrombectomy device 100. In embodiments, coupled to the handle 20, such as within the handle 20, or the like may be any number of motors, pumps 24, etc. for operation of the thrombectomy device 100. In some embodiments, the handle 20 and/or the thrombectomy device 100 may be fluidically coupled to a fluid source 40 (e.g., water, saline, thrombolytic agents, or the like, which may be useful in removal of clot material or other lesion material within a vessel) for supplying fluid through the thrombectomy device 100. In some embodiments, the handle 20 and/or the thrombectomy device 100 may be fluidly coupled to a disposal container 42 for receiving fluid and/or clot particles transported proximally along the thrombectomy device 100. Fluidic coupling may be provided via any number of tubing, piping, etc.

The handle 20 may include any number of user input devices 26, e.g., buttons, toggles, switches, sliders, etc. For operation of the various functionality of the thrombectomy device 100 (e.g., motor operation, pump 24 operation, etc.) In some embodiments, instead of a handle 20, the controller 22, pump 24, etc. may be provided on or within a console to which the thrombectomy device 100 connects.

FIG. 1B generally depicts a longitudinal cross-section of the thrombectomy device 100 taken along line 1B-1B of FIG. 1A. The thrombectomy device 100 is a catheter device formed from a plurality of catheter shafts. In particular, the thrombectomy device 100 generally includes a first catheter shaft 110, a second catheter shaft 120, a third catheter shaft 130, a first helical impeller 136, and a second helical impeller 138. A greater or fewer number of components may be included without departing from the scope of the present disclosure.

The first catheter shaft 110 may extend between a first catheter shaft distal end 111a and a first catheter shaft proximal end 111b (generally indicated in FIG. 1A). The first catheter shaft 110 may define a first lumen 112 extending between the first catheter shaft proximal end 111b and the first catheter shaft distal end 111a. The first catheter shaft 110 may be a flexible tube formed of any number of suitable catheter materials such as but not limited to, rubber, latex, silicone, plastic, PVC, etc. These materials may optionally provide a good balance between flexibility of the catheter, structural integrity, and biocompatibility (e.g. low or zero chemical reactivity with their environment during use). The first catheter shaft 110 has an outer diameter, d₁. As generally depicted in FIG. 1A, the proximal end of the first catheter shaft 110 may be coupled to the fluid source 40 (e.g., reservoir, syringe, etc.) and/or the handle 20.

Still referring to FIG. 1B, the second catheter shaft 120 is positioned radially within the first catheter shaft 110 such that the second catheter shaft 120 is positioned within the first lumen 112. As will be described in greater detail herein, an outflow path 150 configured to direct fluid, such as from the fluid source 40 in a distal direction D, is defined between the first catheter shaft 110 and the second catheter shaft 120. Accordingly, the second catheter shaft 120 may include flexible tube formed, at least partially, of any number of suitable catheter materials such as but not limited to, rubber, latex, silicone, plastic, PVC, etc. The first catheter shaft distal end 111a define an outflow opening 115. For example, the outflow opening 115 may be distally facing. In embodiments, the outflow opening 115 may be positioned concentrically with the longitudinal axis of the thrombectomy device 100. The outflow opening 115 may be generally annular, for example. As will be described in greater detail herein, fluid may flow through the outflow path 150 and out the outflow opening 115.

The second catheter shaft 120 extends between a second catheter shaft distal end 121a and a second catheter shaft proximal end 121b (generally indicated in FIG. 1A). The second catheter shaft 120 defines a second lumen 122 extending between the second catheter shaft distal end 121a and the second catheter shaft proximal end 121b. The second catheter shaft distal end 121a defines an inflow opening 125, such as a plurality of inflow openings. As will be described in greater detail here, the first catheter shaft distal end 111a may be positioned proximal to the second catheter shaft distal end 121a such that the outflow opening 115 defined by the first catheter shaft distal end 111a is positioned proximal to the inflow opening 125.

The third catheter shaft 130 may extend between a third catheter shaft distal end 131a and a third catheter shaft proximal end 131b (generally indicated in FIG. 1A). The third catheter shaft 130 may define a third lumen extending between the third catheter shaft proximal end 131 b and the third catheter shaft distal end 131 a. For example, the third catheter shaft 130 lumen may provide a guidewire lumen 132 for passage of a guidewire 162 through the thrombectomy device 100. An inflow path 152 configured to direct material in a proximal direction P is defined between the second catheter shaft 120 and the third catheter shaft 130. The third catheter shaft 130 may include flexible tube formed of any number of suitable catheter materials such as but not limited to, rubber, latex, silicone, plastic, PVC, etc.

The first helical impeller 136 is positioned within the outflow path 150 and is configured to be moved by fluid flow through the outflow path or to move the fluid through the outflow path 150 to the outflow opening 115 in the distal direction D. The second helical impeller 138 is positioned in the inflow path 152 and is configured to draw fluid released from the outflow opening 115 distally into the inflow opening 125. As depicted, the second helical impeller 138 has a diameter d₂ (e.g., an outer diameter) greater than an inner diameter d₄ of the outflow path 150. That the second helical impeller 138 is larger than the inner diameter d₄ of the outflow path, which may allow the second helical impeller 138 to generate strong proximal fluid flow without enlarging the outflow path or needing the second impeller 138 to be positioned within a narrower part of the inflow path 152 thereby allowing improved flow and control of overall dimensions of the thrombectomy device 100. Each of the helical impellers 136, 138 wrap around the longitudinal axis L within the respective inflow path 152 or outflow path 150. The first helical impeller 136 may be positioned proximal to the second helical impeller 138, such that the first helical impeller 136 and the second helical impeller 138 do not overlap with one another in a radial direction. The second helical impeller 138 is positioned distal to the first helical impeller 136 and is wound in an opposite direction relative to the first helical impeller 136 such that rotation of the second helical impeller 138 generates aspiration into the second lumen 122. For example, the second helical impeller 138 is positioned outside of and distal to the first lumen 112 of the first catheter shaft 110 as depicted.

Each of the first helical impeller 136 and the second helical impeller 138 may include any number of windings (e.g., complete loops or rotations around the longitudinal axis L (e.g., one or more, two or more, three or more, etc. four or less, less than three, less than two, or the like). In embodiments, the first helical impeller 136 and the second helical impeller 138 may have the same number of windings or a different number of windings. To assist in moving fluid in a desired direction, the first helical impeller 136 may be wound in a first direction relative to the longitudinal axis L and the second helical impeller 138 may be wound in a second direction opposite the first direction. That is, for example, the first helical impeller 136 may form a right-handed helix, and the second helical impeller may form a left-handed helix (or vice versa). Accordingly, the first helical impeller 136 may be wound in a direction to direct fluid flow distally through the outflow path 150 and the second helical impeller 138 may be wound in the opposite direction to aspirate fluid into the inflow path 152. That is, during use, both helical impellers 136,138 may rotate in the same rotational direction and, due to the impellers having opposite handedness, rotation of the first helical impeller 136 biases fluid distally out of the outflow path 150 while rotation of the second helical impeller aspirates fluid proximally into the inflow path 152.

Embodiments of the second catheter shaft 120 will now be described in greater detail. Accordingly, and with continued reference to FIG. 1B, the second catheter shaft 120 may include a body portion 124 and a head portion 126 coupled to the body portion 124. In embodiments, a stator 140 connecting the body portion 124 and the head portion 126 is also depicted. The body portion 124 may generally be a flexible tube such as formed of any suitable material, such as, but not limited to, rubber, latex, silicone, plastic, PVC, etc. The body portion 124 may be positioned within the first lumen 112, as depicted. The body portion 124 has a first diameter d₄.

In the illustrated embodiment, the head portion 126 is coupled to the body portion 124 of the second catheter shaft 120 such that the head portion 126 is at least partially positioned outside and distal to the first catheter shaft 110. The head portion 126 may be formed of a rigid material, such as a material more rigid than the body portion 124 (e.g., plastic, metal, such as stainless steel, or the like suitable for positioning within a body vessel). The head portion 126 may have an outer profile that is at least partially tapered toward the second catheter shaft distal end 121a, which may provide improved maneuverability of the thrombectomy device 100 within a vessel (e.g., an artery or a vein). The head portion 126 may have a maximum outer diameter d₅, which is larger than the diameter d₄ of the body portion 124.

Referring briefly to FIGS. 1C and 1D, the head portion 126 may define the inflow opening 125 providing an inlet into the second lumen 122 and the inflow path 152. For example, the inflow opening 125 may be formed through a tapered surface 128 of the head portion 126. It is noted that though four openings are illustrated, there may be any number of inflow openings (e.g., one or more, two or more, three or more, etc.). The inflow opening 125 may be elongated and/or may be any suitable shape for aspiration of clot material (e.g., oval, rectangular, or any regular or irregular polygonal or non-polygonal shape).

In embodiments, the head portion 126 may define a guidewire passage 127. For example, the guidewire passage 127 may be positioned within a tip of the head portion 126, such as centrally in the tip so as to be concentric with the longitudinal axis L of the thrombectomy device 100, such as illustrated in FIG. 1B. In embodiments, the inflow opening 125 may include a plurality of inflow openings equably arranged around the guidewire passage 127, as illustrated in FIG. 1D. In embodiments, a guidewire 162 is positionable within the third lumen and extends out of the thrombectomy device 100 through the guidewire passage 127, as schematically depicted in FIG. 1B.

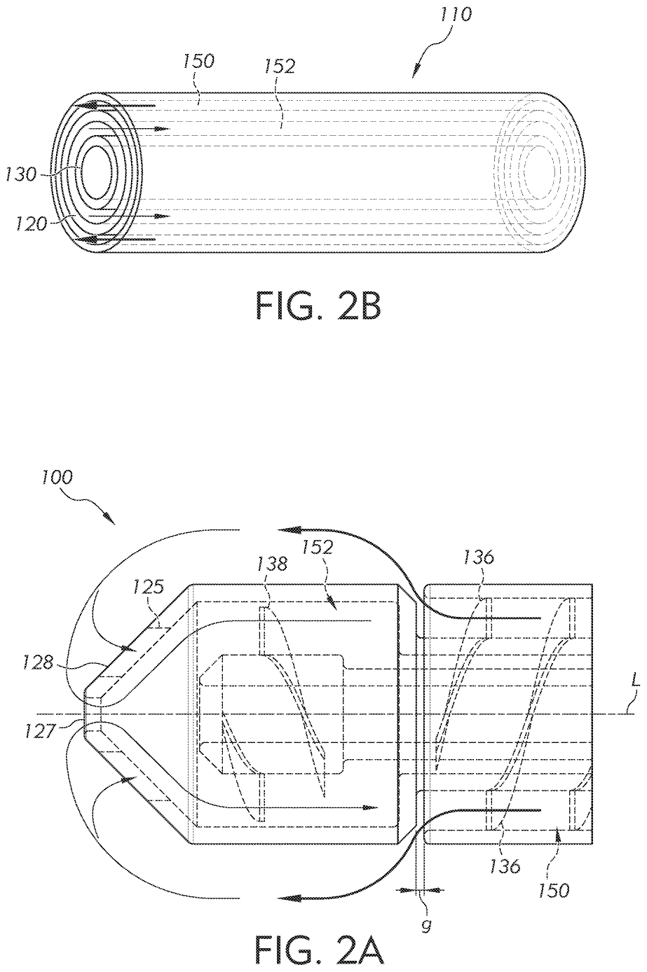

In the illustrated embodiment, the head portion 126 includes a first portion 142 that is positioned within the first lumen 112 and a second portion 148 that is positioned outside of the first lumen 112. That is the first portion 142 may have a smaller diameter than the second portion 148 to fit within the first lumen 112 without blocking the inflow path 152. The first portion 142 may have a diameter (e.g., the maximum outer diameter ds noted above) substantially equal to or less than the outer diameter di of the first catheter shaft 110. In embodiments, the head portion 126 is spaced from the first catheter shaft distal end 111a of the first catheter shaft 110 by a gap distance g (shown in FIG. 2A). For example, the second portion 148 may be longitudinally spaced from the first catheter shaft distal end 111a by the gap, thereby providing access for fluid to leave the outflow path 150 via the outflow opening 115.

Referring to FIGS. 1B and 1C, in the illustrated embodiment, the first helical impeller 136 is coupled to the first portion 142 of the head portion 126 and the second helical impeller 138 is coupled to the second portion 148 within the second lumen 122. For example, the first helical impeller 136 and the second helical impeller 138 may be coupled to the head portion 126 via welding, soldering, adhesive coupled, or the like. Alternatively, the first helical and/or the second helical impeller 136, 138 may be integrally formed with the head portion 126 (e.g., via machining, molding, etc.) In embodiments, the head portion 126, the first helical impeller 136, and/or the second helical impeller 138 may be formed of metal, such as stainless steel, plastic, and/or any suitable rigid material for insertion into a vessel.

Referring specifically to FIG. 1B, the head portion 126 may be coupled to the body portion 124 via the stator 140. For example, the stator 140 may be a tube, which may couple the body portion 124 to the head portion 126 and support rotation of the head portion 126 relative to the stator 140 and the body portion 124. The stator 140 may be formed of metal, such as stainless steel, plastic, and/or any suitable rigid material for insertion into a vessel. In embodiments, the stator 140 may include a grooved proximal end 141 configured to be received within the body portion 124 and affixed thereto (e.g., via adhesive, press fit, etc.). The stator 140 may further include a flanged distal end 143. The flanged distal end 143 may engage with second portion 148 of the head portion 126 such as along an engagement surface 186 and have a greater diameter than the first portion 142, such as to prevent the head portion 126 from slipping off of the stator 140. Accordingly, the first portion 142, the stator 140, and the body portion 124 may be positioned within the first lumen 112.

At the first catheter shaft distal end 111a of the first catheter shaft 110 may be an internal collar 160. The internal collar 160 may act as a bearing surface and may be in contact with the first helical impeller 136 and support rotation of the first helical impeller 136. The internal collar 160 may be grooved or slotted so as to be received within the first lumen 112 and coupled thereto (e.g., via welding, brazing, adhesive, or the like). The internal collar 160 may be formed of any suitable material such as, a polymer blend such as Pebax or Grilamid, or a polyamide, or the like. The internal collar 160 may optionally provide a predefined shape for a passage within which the first helical impeller 136 rotates during use. This may optionally make it easier for the first helical impeller to rotate with minimal friction, and to minimize fluid leakage between the internal collar 160 and first helical impeller 136.

Still referring to FIG. 1B, the third catheter shaft 130 may include an enlarged diameter portion 134 at the third catheter shaft distal end 131a relative to a more proximal portion of the third catheter shaft 130. In some embodiments, the enlarged diameter portion 134 may be a separate component coupled to the more proximal portion of the third catheter shaft 130, such as via a press fit, adhesive, or the like. In some embodiments, the enlarged diameter portion 134 may be integrally formed with the more proximal portion of the third catheter shaft 130, thereby reducing components. It is contemplated that the enlarged diameter portion 134 may provide a stator against which the second helical impeller 138 may rotate. This enlarged diameter portion 138 may optionally help to reduce leakage at an inner diameter of the second helical impeller 138. The enlarged diameter portion 134 may further provide centering to the head portion 126 and the second helical impeller 138 so as to maintain axial alignment of the head portion 126 and the second helical impeller 138. The enlarged diameter portion 134 may be formed of any suitable material, such as a polymer blend such as Pebax or Grilamid, or a polyamide, or the like.

In embodiments, fluid may be pushed into the outflow path 150 via a pump 24 (or similar device). The pump 24 may fluidically couple the outflow path 150 to a fluid source 40 (e.g., water, saline, thrombolytic, or the like). As noted above, the pump 24 may be within the handle 20 or otherwise operatively coupled to the outflow path 150, such as through the handle 20. As generally illustrated in FIGS. 2A and 2B, the pump 24 (schematically depicted in FIG. 1a) pushes fluid through the outflow path 150, which in turn contacts the first helical impeller 136, causing the first helical impeller 136 to rotate about the longitudinal axis L. That is, the fluid acts as a liquid clutch to cause the first helical impeller 136 and/or the head portion 126 (supported by bearing 160) to rotate, which in turn may also cause the second helical impeller 138 to rotate. Accordingly, rotation of the two impellers 136, 138 may be achieved without need for a motor. As the second helical impeller 138 rotates, and because the second helical impeller 138 is wound in an opposite direction from the first helical impeller 136 (e.g. has a right-handed winding vs. a left-handed winding for the first helical impeller 136, or vice versa), the second helical impeller 138 produces suction to draw the fluid released from the outflow path 150 distally toward the inflow opening 125 (and any clot positioned close to the inflow opening 125). Accordingly, fluid is substantially removed from the vessel without dwelling therein for a substantial period of time, leading to reduced bleeding, hospital stays, etc. That is, if a thrombectomy fluid is flowed through the outflow opening 115, the fluid may contact the thrombus, but may be promptly removed by being aspirated into the second lumen. The fluid may be drawn into the inflow opening 125 (and in some cases even the guidewire passage 127) of the head portion 126 and then directed through the inflow path 152 as further depicted in FIG. 2B. The aspiration along with the fluid delivery may allow for breakup of clots and removal thereof via the thrombectomy device 100. Accordingly, the thrombectomy device 100 may be operated without a motor to perform a thrombectomy procedure.

In various embodiments, the thrombectomy device 100 may operate without a vacuum pump or other source of vacuum beyond that created locally by the second helical impeller 138. The lack of need of a vacuum pump and/or a motor may provide a simpler device for performing a thrombectomy procedure with less set up. However, in any of the embodiments described herein, the thrombectomy device 100, such as the second catheter shaft 120, may be fluidically coupled to a vacuum source (e.g., a vacuum pump or other source). For example, the vacuum source may be positioned within the handle 20 or otherwise fluidically couplable to the thrombectomy device 100. Additionally, in any of the embodiments described herein, the second catheter shaft 120 and/or the head portion 126 may be operatively coupled to a motor or similar rotational actuator to directly drive rotation of the first and second helical impeller 138. Actively driving movement of the first and second impellers 136, 138 may provide for stronger flows and improved clot maceration. In such embodiments, a pump and/or vacuum may not be needed to move fluid through the thrombectomy device 100, which may reduce weight and/or complexity.

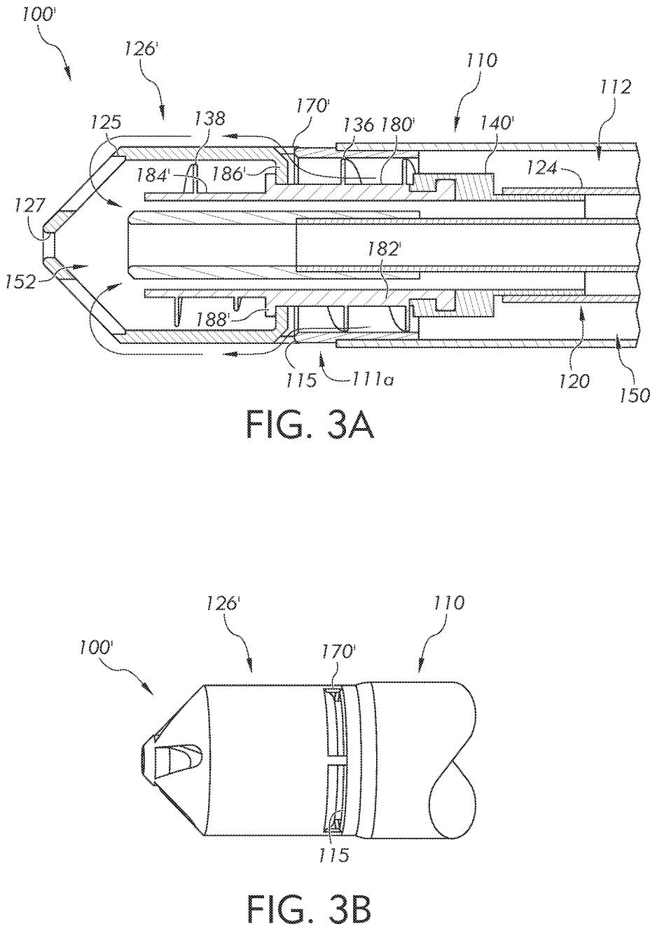

Referring now to FIGS. 3A and 3B, another embodiment of the thrombectomy device 100′ is illustrated. The thrombectomy device 100′ is substantially similar to the embodiments as described. Accordingly, the above description applies unless otherwise noted or apparent. In the present embodiment, coupled to the first catheter shaft distal end 111a is a head portion 126′, similar to the head portion 126 described above with respect to FIGS. 1-3. In this embodiment, the head portion 126′ defines the distal end of the thrombectomy device 100′, and as above, may define one or more inflow openings 125 and/or a guidewire passage 127. However, in the present embodiment, the head portion 126′ only includes a second portion (as opposed to both the first portion and the second portion described further above) and does not include a helical impeller coupled thereto. The head portion 126′ is rigidly connected to the first catheter shaft 110, such as at the first catheter shaft distal end 111a (e.g., to the internal collar 160). For example, the head portion 126′ may be rigidly coupled to the first catheter shaft 110 via one or more connector bridges 170′. The one or more connector bridges 170′ may be small pieces or strips of material that connect the head portion 126′ to the first catheter shaft distal end 111a. Accordingly, the one or more connector bridges 170′ may connect the head portion 126 to the first catheter shaft distal end 111a, such as via welding braising, adhesive, etc. The one or more connector bridges 170′ extend across the outflow opening 115, and may be intermittently placed or otherwise spaced apart from one another so as not to completely or substantially occlude the outflow opening 115.

The second catheter shaft 120 of the present embodiment may include a helix carrier 180′ and, in some embodiments, a stator 140′ coupled to the body portion 124, the body portion 124 being substantially similar to the body portion 124 described in greater detail above.

The helix carrier 180′ may be a tube coupled to the body portion 124 (e.g., via the stator 140′ or directly such as via welding, press fits, adhesives, or the like). The helix carrier 180′ may include a first carrier portion 182′, which is coupled to the first helical impeller 136, and a second carrier portion 184′ coupled to the second helical impeller 138, the first and second helical impellers 136, 138 may be as described in the above embodiments. The helix carrier 180′ may carry the first helical impeller 136 on an outer diameter of the first carrier portion 182′ and the second helical impeller 138 on an outer diameter of the second carrier portion 184′. The helical impellers 136 may be coupled to the helix carrier 180′ via welding, brazing, glue, or the like. Accordingly, the helix carrier may be formed of metal, plastic, or the like and may be formed of same or similar material to the head portion 126′, though it is contemplated the helix carrier 180′ may be formed of a different material from the head portion 126′.

The first carrier portion 182′ with the first helical impeller 136 may positioned within the first lumen 112 proximal to the head portion 126. The second carrier portion 184′ along with the second helical impeller 138 may be positioned within the head portion 126′ distal to the outflow opening 115. In some embodiments, the helix carrier 180′ further includes a flange 188′ between the first carrier portion 182′ and the second carrier portion 184′, engaged with an engagement surface 186′ of the head portion 126′. The helix carrier 180′ is rotatable such that the flange 188′ may rotate against or across the engagement surface 186′ of the head portion 126.

Similar to the embodiment described above, as fluid flows through the outflow path 150, such as via operation of a pump 24, syringe, or the like fluidically coupled to the outflow path 150, the fluid may impinge upon the first helical impeller 136 causing it to rotate. Accordingly, the fluid impact causes the helix carrier 180′ to rotate, which in turn causes the second helical impeller 138 to rotate to draw or aspirate fluid into the inflow path 152 thereby allowing for fluid removal through the inflow path 152. However, as in the embodiment described above, a motor may be operatively coupled to the second catheter shaft 120 and/or the helix carrier 180′ to drive rotation of the first and second helical impellers 136, 138.

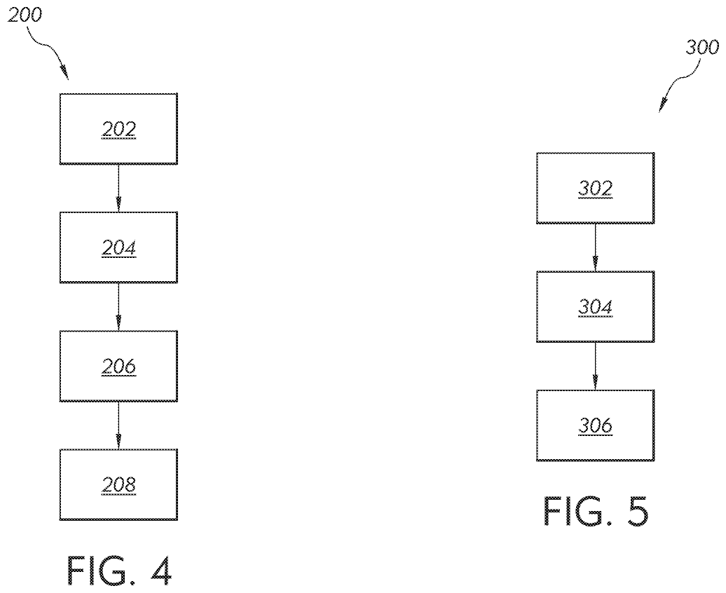

Referring now to FIG. 4, a flowchart illustrating a method 200 of assembling a thrombectomy device 100 according to the various embodiments shown and described herein. It is noted that though the steps are recited in relation to the embodiment depicted in FIG. 1A-2B, similar steps would be applicable to the embodiment depicted in FIG. 4. A greater or fewer number of steps may be included and the steps may be performed in any order.

With reference also to FIG. 1B, at block 202, the method 200 includes positioning the first catheter shaft 110 about the second catheter shaft 120 such that the second catheter shaft 120 is positioned within the first lumen 112, thereby defining the outflow path 150 configured to direct fluid in the distal direction between the first catheter shaft 110 and the second catheter shaft 120. For example, block 202 may include positioning the body portion 124 of the second catheter shaft 120 within the first lumen 112 of the first catheter shaft 110. Further block 202 may include positioning the head portion 126 of the second catheter shaft 120 outside and distal to the first catheter shaft 110. At block 204, the method 200 further includes positioning the third catheter shaft 130 within the second lumen 122 such that the inflow path 152 configured to direct material in a proximal direction is defined between the second catheter shaft 120 and the third catheter shaft 130. In embodiments, positioning the head portion 126 of the second catheter shaft 120 outside and distal to the first catheter shaft 110 may include creating a gap distance g (shown in FIG. 2A) between the first catheter shaft distal end 111a of the first catheter shaft 110 and the head portion 126 of the second catheter shaft 120, thereby providing space for the fluid to flow out of the outflow opening 115.

At block 206, the method 200 further includes positioning a first helical impeller 136 in the outflow path 150, such that the first helical impeller 136 is oriented to direct the fluid through the outflow path 150 to the outflow opening 115. For example, the first helical impeller 136 may the welded, adhered, brazed, or the like to the second catheter shaft 120 or to a head portion 126 as describe herein to traverse the outflow path 150 and encircle the longitudinal axis. Similarly, at block 208 the method 200 further includes positioning the second helical impeller 138 within the inflow path 152 such that the second helical impeller 138 is oriented to draw the fluid released from the outflow opening 115 distally into the inflow opening 125. For example, the second helical impeller 138 may the welded, adhered, brazed, or the like to the second catheter shaft 120 or to a head portion 126 as describe herein to traverse the inflow path 152 and encircle the longitudinal axis. For example, the block 208 may include positioning the second helical impeller 138 within the head portion 126 of the second catheter shaft 120 and/or positioning the second helical impeller 138 outside of and distal to the first lumen 112 of the first catheter shaft 110.

Referring now to FIG. 5 a flowchart depicting a method 300 of using a thrombectomy device 100 is generally depicted. The method 300 may include a greater or fewer number of steps, and the steps may be performed in any order.

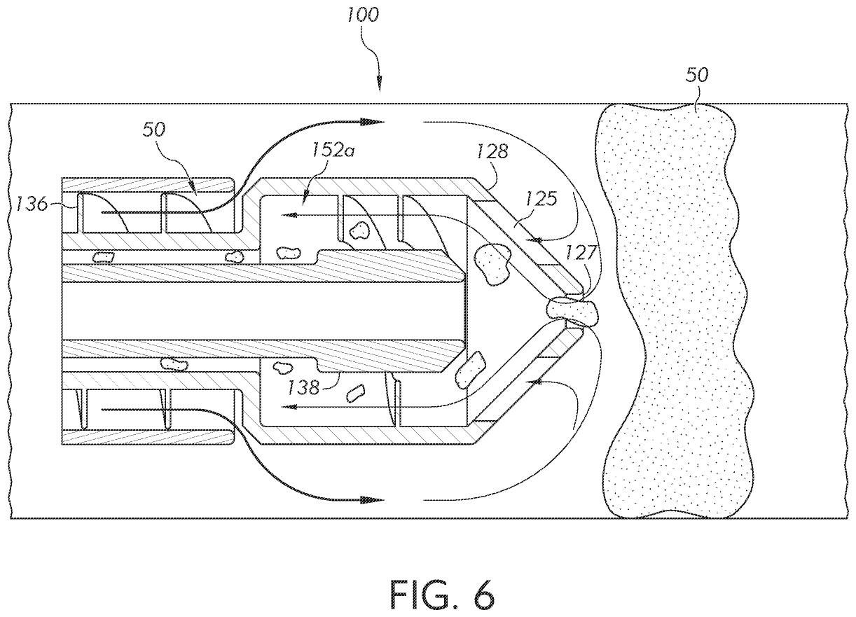

Referring to block 302, the method 300 includes positioning the thrombectomy site at a target site. For example, a target site may be positioned within a vessel where there is a clot 50. For example, FIG. 6 illustrates a thrombectomy device 100 (though thrombectomy device 100′ may instead be used with any of the steps described herein) at a target site including a clot 50 within a vessel (e.g., a vein or artery). The method 300 at block 304 includes rotating the first helical impeller 136 to move the fluid in the distal direction through the outflow path 150. The method 300 at block 306 includes rotating the second helical impeller 138 to initiate suction through the inflow path 152. In embodiments, the method 300 further includes drawing fluid released from the outflow path 150 distally into the inflow path 152 via suction generated by the second helical impeller 138. The combination of delivering fluid through the outflow opening 115 and drawing it distally through the vessel into contact with the clot 50 and then into the inflow path 152 creates a local vortex to assist with macerating the target lesion (in this case clot 50). Additionally, the method 300 may include removing the fluid (for example a thrombolytic) quickly, thereby reducing unintentional bleeding which may otherwise be caused through traditional thrombolytic use may be avoided, leading to improved recovery.

Embodiments of the present disclosure may be further described with reference to the following numbered clauses:

1. A thrombectomy device comprising: a first catheter shaft defining a first lumen and comprising a first catheter shaft distal end; a second catheter shaft positioned within the first lumen, wherein an outflow path configured to direct fluid in a distal direction is defined between the first catheter shaft and the second catheter shaft, the second catheter shaft defining a second lumen and comprising a second catheter shaft distal end defining an inflow opening, wherein the first catheter shaft distal end defines an outflow opening positioned proximal to the inflow opening; a third catheter shaft positioned within the second lumen, wherein an inflow path configured to direct material in a proximal direction is defined between the second catheter shaft and the third catheter shaft; a first helical impeller positioned in the outflow path configured to direct the fluid through the outflow path to the outflow opening; and a second helical impeller positioned within the inflow path, the second helical impeller configured to draw the fluid released from the outflow opening distally into the inflow opening, the second helical impeller having a diameter greater than an inner diameter of the outflow path.

2. The thrombectomy device of clause 1, wherein: the first catheter shaft has an outer diameter; and the second catheter shaft has a body portion positioned within the first lumen, the body portion having a first diameter, and a head portion positioned outside and distal to the first catheter shaft, the head portion coupled to the body portion and having a maximum outer diameter greater than the first diameter.

3. The thrombectomy device of clause 2, wherein the second helical impeller is within the head portion of the second catheter shaft.

4. The thrombectomy device of any preceding clause, wherein the maximum outer diameter of the head portion is less than or equal to the outer diameter of the first catheter shaft.

5. The thrombectomy device of any preceding clause, wherein the head portion is spaced from the first catheter shaft distal end of the first catheter shaft by a gap distance.

6. The thrombectomy device of any preceding clause, wherein the second helical impeller is positioned outside of and distal to the first lumen of the first catheter shaft.

7. The thrombectomy device of any preceding clause, wherein the first helical impeller is wound in a first direction relative to a longitudinal axis and the second helical impeller is wound in a second direction opposite the first direction.

8. A thrombectomy device comprising: a first catheter shaft defining a first lumen and comprising a first catheter shaft distal end; a second catheter shaft positioned within the first lumen, wherein an outflow path configured to direct fluid in a distal direction is defined between the first catheter shaft and the second catheter shaft, the second catheter shaft defining a second lumen and comprising a second catheter shaft distal end defining an inflow opening, wherein the first catheter shaft distal end defines an outflow opening positioned proximal to the inflow opening; a third catheter shaft positioned within the second lumen, the third catheter shaft defining a guidewire lumen, wherein an inflow path configured to direct material in a proximal direction is defined between the second catheter shaft and the third catheter shaft; a first helical impeller positioned in the outflow path configured to direct the fluid through the outflow path to the outflow opening; a second helical impeller positioned within the inflow path, the second helical impeller configured to draw the fluid released from the outflow opening distally into the inflow opening, the second helical impeller having a diameter greater than an inner diameter of the outflow path; and a guidewire configured to be positioned within the guidewire lumen.

9. The thrombectomy device of any preceding clause, wherein: the first catheter shaft has an outer diameter; and the second catheter shaft has a body portion positioned within the first lumen, the body portion having a first diameter, and a head portion positioned outside and distal to the first catheter shaft, the head portion coupled to the body portion and having a maximum outer diameter greater than the first diameter.

10. The thrombectomy device of any preceding clause, wherein the second helical impeller is within the head portion of the second catheter shaft.

11. The thrombectomy device of any preceding clause, wherein the maximum outer diameter of the head portion is less than or equal to the outer diameter of the first catheter shaft.

12. The thrombectomy device of any preceding clause, wherein the head portion is spaced from the first catheter shaft distal end of the first catheter shaft by a gap distance.

13. The thrombectomy device of any preceding clause, wherein the second helical impeller is positioned outside of and distal to the first lumen of the first catheter shaft.

14. The thrombectomy device of any preceding clause, wherein the first helical impeller is wound in a first direction relative to a longitudinal axis and the second helical impeller is wound in a second direction opposite the first direction.

15. A method of assembling a thrombectomy device comprising: positioning a first catheter shaft defining a first lumen and comprising a first catheter shaft distal end about a second catheter shaft such that the second catheter shaft is positioned within the first lumen, wherein an outflow path configured to direct fluid in a distal direction is defined between the first catheter shaft and the second catheter shaft, the second catheter shaft defining a second lumen and comprising a second catheter shaft distal end defining an inflow opening, wherein the first catheter shaft distal end defines an outflow opening positioned proximal to the inflow opening; positioning a third catheter shaft within the second lumen, wherein an inflow path configured to direct material in a proximal direction is defined between the second catheter shaft and the third catheter shaft; positioning a first helical impeller in the outflow path, the first helical impeller configured to direct the fluid through the outflow path to the outflow opening; and positioning a second helical impeller within the inflow path, the second helical impeller configured to draw the fluid released from the outflow opening distally into the inflow opening, the second helical impeller having a diameter greater than an inner diameter of the outflow path.

16. The method of any preceding clause, wherein positioning the first catheter shaft about the second catheter shaft comprises: positioning a body portion of the second catheter shaft within the first lumen, the body portion having a first diameter; and positioning a head portion of the second catheter shaft outside and distal to the first catheter shaft, the head portion coupled to the body portion and having a maximum outer diameter greater than the first diameter.

17. The method any preceding clause, wherein positioning the second helical impeller within the inflow path comprises positioning the second helical impeller is within the head portion of the second catheter shaft.

18. The method of any preceding clause, wherein positioning the head portion of the second catheter shaft outside and distal to the first catheter shaft comprising creating a gap distance between the first catheter shaft distal end of the first catheter shaft and the head portion of the second catheter shaft.

19. The method of any preceding clause, wherein positioning the second helical impeller comprises positioning the second helical impeller outside of and distal to the first lumen of the first catheter shaft.

20. A method of using a thrombectomy device comprising: positioning the thrombectomy device at a target site, the thrombectomy device comprising: a first catheter shaft defining a first lumen and comprising a first catheter shaft distal end; a second catheter shaft positioned within the first lumen, wherein an outflow path configured to direct fluid in a distal direction is defined between the first catheter shaft and the second catheter shaft, the second catheter shaft defining a second lumen and comprising a second catheter shaft distal end defining an inflow opening, wherein the first catheter shaft distal end defines an outflow opening positioned proximal to the inflow opening; a third catheter shaft positioned within the second lumen, wherein an inflow path configured to direct material in a proximal direction is defined between the second catheter shaft and the third catheter shaft; a first helical impeller positioned in the outflow path configured to direct the fluid through the outflow path to the outflow opening; and a second helical impeller positioned within the inflow path, the second helical impeller configured to draw the fluid released from the outflow opening distally into the inflow opening, the second helical impeller having a diameter greater than an inner diameter of the outflow path; rotating the first helical impeller to move the fluid in the distal direction through the outflow path; and rotating the second helical impeller to initiate suction through the inflow path.

21. The method of any preceding clause, further comprising drawing fluid released from the outflow path distally into the inflow path via suction generated by the second helical impeller.

22. The method of any preceding clause, wherein the fluid is a thrombolytic.

23. A method of using the thrombectomy device of any of clauses 1-14 comprising: positioning the thrombectomy device at a target site; rotating the first helical impeller to move the fluid in the distal direction through the outflow path; and rotating the second helical impeller to initiate suction through the inflow path.

24. A method of assembling the thrombectomy device of any of clauses 1-14, comprising: positioning the second catheter shaft within the first catheter shaft and positioning the third catheter shaft within the second catheter shaft.

It should now be understood that embodiments as described herein are directed to improved thrombectomy devices and methods. In particular, thrombectomy devices as described herein, include helical impellers that are operated to push fluid out the outflow path and pull fluid and/or material into the inflow path, thereby creating a local vortex operable to macerate clot material and remove the clot or other targeted lesion via the inflow path.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A thrombectomy device comprising:
a first catheter shaft defining a first lumen and comprising a first catheter shaft distal end;
a second catheter shaft positioned within the first lumen, wherein an outflow path configured to direct fluid in a distal direction is defined between the first catheter shaft and the second catheter shaft, the second catheter shaft defining a second lumen and comprising a second catheter shaft distal end defining an inflow opening, wherein the first catheter shaft distal end defines an outflow opening positioned proximal to the inflow opening;
a third catheter shaft positioned within the second lumen, wherein an inflow path configured to direct material in a proximal direction is defined between the second catheter shaft and the third catheter shaft;
a first helical impeller positioned in the outflow path configured to direct the fluid through the outflow path to the outflow opening; and
a second helical impeller positioned within the inflow path, the second helical impeller configured to draw the fluid released from the outflow opening distally into the inflow opening, the second helical impeller having a diameter greater than an inner diameter of the outflow path.

2. The thrombectomy device of claim 1, wherein:
the first catheter shaft has an outer diameter; and
the second catheter shaft has a body portion positioned within the first lumen, the body portion having a first diameter, and a head portion positioned outside and distal to the first catheter shaft, the head portion coupled to the body portion and having a maximum outer diameter greater than the first diameter.

3. The thrombectomy device of claim 2, wherein the second helical impeller is within the head portion of the second catheter shaft.

4. The thrombectomy device of claim 2, wherein the maximum outer diameter of the head portion is less than or equal to the outer diameter of the first catheter shaft.

5. The thrombectomy device of claim 2, wherein the head portion is spaced from the first catheter shaft distal end of the first catheter shaft by a gap distance.

6. The thrombectomy device of claim 2, wherein the second helical impeller is positioned outside of and distal to the first lumen of the first catheter shaft.

7. The thrombectomy device of claim 1, wherein the first helical impeller is wound in a first direction relative to a longitudinal axis and the second helical impeller is wound in a second direction opposite the first direction.

8. A thrombectomy device comprising:
a first catheter shaft defining a first lumen and comprising a first catheter shaft distal end;
a second catheter shaft positioned within the first lumen, wherein an outflow path configured to direct fluid in a distal direction is defined between the first catheter shaft and the second catheter shaft, the second catheter shaft defining a second lumen and comprising a second catheter shaft distal end defining an inflow opening, wherein the first catheter shaft distal end defines an outflow opening positioned proximal to the inflow opening;
a third catheter shaft positioned within the second lumen, the third catheter shaft defining a guidewire lumen, wherein an inflow path configured to direct material in a proximal direction is defined between the second catheter shaft and the third catheter shaft;
a first helical impeller positioned in the outflow path configured to direct the fluid through the outflow path to the outflow opening;
a second helical impeller positioned within the inflow path, the second helical impeller configured to draw the fluid released from the outflow opening distally into the inflow opening, the second helical impeller having a diameter greater than an inner diameter of the outflow path; and
a guidewire configured to be positioned within the guidewire lumen.

9. The thrombectomy device of claim 8, wherein:
the first catheter shaft has an outer diameter; and
the second catheter shaft has a body portion positioned within the first lumen, the body portion having a first diameter, and a head portion positioned outside and distal to the first catheter shaft, the head portion coupled to the body portion and having a maximum outer diameter greater than the first diameter.

10. The thrombectomy device of claim 9, wherein the second helical impeller is within the head portion of the second catheter shaft.

11. The thrombectomy device of claim 9, wherein the maximum outer diameter of the head portion is less than or equal to the outer diameter of the first catheter shaft.

12. The thrombectomy device of claim 9, wherein the head portion is spaced from the first catheter shaft distal end of the first catheter shaft by a gap distance.

13. The thrombectomy device of claim 9, wherein the second helical impeller is positioned outside of and distal to the first lumen of the first catheter shaft.

14. The thrombectomy device of claim 8, wherein the first helical impeller is wound in a first direction relative to a longitudinal axis and the second helical impeller is wound in a second direction opposite the first direction.

15. A method of assembling a thrombectomy device comprising:
positioning a first catheter shaft defining a first lumen and comprising a first catheter shaft distal end about a second catheter shaft such that the second catheter shaft is positioned within the first lumen, wherein an outflow path configured to direct fluid in a distal direction is defined between the first catheter shaft and the second catheter shaft, the second catheter shaft defining a second lumen and comprising a second catheter shaft distal end defining an inflow opening, wherein the first catheter shaft distal end defines an outflow opening positioned proximal to the inflow opening;
positioning a third catheter shaft within the second lumen, wherein an inflow path configured to direct material in a proximal direction is defined between the second catheter shaft and the third catheter shaft;
positioning a first helical impeller in the outflow path, the first helical impeller configured to direct the fluid through the outflow path to the outflow opening; and
positioning a second helical impeller within the inflow path, the second helical impeller configured to draw the fluid released from the outflow opening distally into the inflow opening, the second helical impeller having a diameter greater than an inner diameter of the outflow path.

16. The method of claim 15, wherein positioning the first catheter shaft about the second catheter shaft comprises:
positioning a body portion of the second catheter shaft within the first lumen, the body portion having a first diameter; and
positioning a head portion of the second catheter shaft outside and distal to the first catheter shaft, the head portion coupled to the body portion and having a maximum outer diameter greater than the first diameter.

17. The method of claim 16, wherein positioning the second helical impeller within the inflow path comprises positioning the second helical impeller is within the head portion of the second catheter shaft.

18. The method of claim 16, wherein positioning the head portion of the second catheter shaft outside and distal to the first catheter shaft comprising creating a gap distance between the first catheter shaft distal end of the first catheter shaft and the head portion of the second catheter shaft.

19. The method of claim 16, wherein positioning the second helical impeller comprises positioning the second helical impeller outside of and distal to the first lumen of the first catheter shaft.

20. A method of using the thrombectomy device of claim 1, comprising:
positioning the thrombectomy device at a target site;
rotating the first helical impeller to move the fluid in the distal direction through the outflow path; and
rotating the second helical impeller to initiate suction through the inflow path.

21-22. (canceled)
